(19) United States
(12) Patent Application Publication
(10) Pub. No.: US 2026/0225590 A1
(43) Pub. Date: Aug. 6, 2026

(54) METHOD FOR GENERATING TRAINING DATA FOR A SOFTWARE AGENT, METHOD FOR TRAINING A SOFTWARE AGENT, METHOD FOR ASSISTING A DRIVER IN THE CONTROL OF A MOTOR VEHICLE, AND MOTOR VEHICLE

(71) Applicant: VOLKSWAGEN AKTIENGESELLSCHAFT, Wolfsburg (DE)

(72) Inventors: Samuel Schacher, Berlin (DE); Amira El Amouri, Wolfsburg (DE)

(21) Appl. No.: 19/153,927

(22) PCT Filed: Jan. 9, 2024
(86) PCT No.: PCT/EP2024/050343

§ 371 Date: Aug. 5, 2025

(30) Foreign Application Priority Data
Feb. 9, 2023 (DE) 102023201074.7

(51) Int. Cl. B60W 30/095; B60W 60/00; G06F 18/214

(52) U.S. Cl. CPC B60W 30/095; B60W 60/0016; G06F 18/214

(57) ABSTRACT

Technologies and techniques for generating training data for a software agent configured to control a driver assistance function of a motor vehicle. A trajectory is planned for a detected driving situation using the driver assistance function. Longitudinal and/or lateral control for guiding the motor vehicle along the trajectory is also planned, and an actuator system of the vehicle is adjusted to implement the control. A response of the driver to the adjusted actuator system is determined, and a divergence value is calculated based on the difference between the control indicated by the driver's response and the control performed by the driver assistance function. The driving situation is then provided, together with the associated divergence value, as training data for the software agent. This allows the software agent to be trained in a manner that reduces divergence and improves alignment between the driver assistance function and the driver's control preferences.

### RELATED APPLICATIONS

The present application claims priority to International Patent Application No. PCT/EP2024/050343 to Schacher, et al., filed Jan. 9, 2024, which claims priority from German Patent App. No. DE 10 2023 201 074.7, filed Feb. 9, 2023, the contents of each being incorporated by reference in their entirety herein.

### TECHNICAL FIELD

The present disclosure is related to technologies and techniques for generating training data for a software agent, training a software agent, for assisting a driver in the control of a motor vehicle by means of a driver assistance function, and to a motor vehicle comprising a driver assistance system.

### BACKGROUND

DE 10 2018 202 146 A1 discloses a method for selecting a driving profile of a motor vehicle, a driver assistance system for this purpose, and a motor vehicle equipped with it. It is provided that a deviation between the driving profile currently requested by the driver and one from several specified driving profiles that was used previously is recognized based on an action taken by the driver. After the deviation is recognized, a query is presented to the driver asking whether the deviation should be learned by the driver assistance system for the current situation. Once the driver confirms the query, the driver assistance system is adapted to the current situation in at least one parameter influencing the selection of the driving profile to be used, so that the deviation is appropriately considered during the automatic selection of the driving profile for future situations that correspond to the current situation.

Furthermore, CN 109 927 725 A discloses an adaptive cruise control system that can learn a driving style.

In addition, a learning method is known from EP 3 690 769 A1 for acquiring at least one personalized reward function, which is used for executing a Reinforcement Learning algorithm and which corresponds to a personalized optimal strategy for a driver.

Driver assistance systems and functions of automated driving are progressively taking over tasks traditionally performed by human drivers. As a result, they introduce their own preprogrammed driving style and driving decisions into the driving task. In this context, driving style primarily describes aspects that, although influenced by external boundary conditions such as traffic situation, weather, time, or driver fitness, are more attributable to personal characteristics, such as speed and lane selection, acceleration and braking behavior, the manner in which a traffic circle is navigated, preferred distance from the side of the road, coasting in front of red traffic lights, or entering towns on rural roads. In this context, driving decisions primarily describe aspects of passing, lane changes, merging maneuvers, general interactions in road traffic, and the behavior of how closely a vehicle is approached from behind before action is taken. When these preprogrammed characteristics diverge from the driver's style and the personal or subjective decisions of the driver using the system, it may result in poor ratings, all the way to deactivation or non-use of these driver assistance systems. One of the greatest challenges, particularly with driver assistance systems but also with fully automatic operation, is for the customer to evaluate the system positively and demonstrate a willingness to actually use it. Multiple levers exist in this regard, with a central lever being the divergence, or ideally the non-existence of divergence, between the driver's driving style and the situation-based decisions of the driver assistance system.

### SUMMARY

Aspects of the present disclosure are directed to creating solutions that makes it possible to adapt a driver assistance function particularly well to a driver of a motor vehicle.

Some aspects are achieved by the subject matter of the independent claims. Further aspects are disclosed in the dependent claims, the description, and the figures. Features, advantages and possible embodiments that are set out within the scope of the description for one of the subjects of the independent claims are to be regarded at least analogously as features, advantages and possible embodiments of the particular subject matter of the other independent claims as well as any possible combination of the subjects of the independent claims, possibly in conjunction with one or more of the subclaims.

In some examples, a method is disclosed for generating training data for a software agent, which is configured to control a driver assistance function of a motor vehicle. A driver of the motor vehicle can be supported in certain driving situations by means of the driver assistance function, for example by controlling an actuator system of the motor vehicle. The driver can be supported by means of the driver assistance function during the lateral guidance and/or during the longitudinal guidance of the motor vehicle.

In some examples, an electronic processing device is disclosed, configured to carry out the method for generating the training data for the software agent. The electronic processing device can be configured to control the driver assistance function and/or to trigger the setting of the actuator system. The electronic processing device can furthermore be configured to ascertain the reaction of the driver and, in turn, to ascertain the divergence characterized by the divergence value based on the ascertained reaction. Furthermore, the electronic processing device can be configured to generate the training data from the ascertained situation, together with the ascertained assigned divergence value, and to provide these to the software agent.

In some examples, a method for training a software agent is disclosed, comprising an artificial neural network, using training data generated using any of the methods disclosed herein. By training the software agent, the software agent can control the driver assistance function in a manner that is adapted particularly well to a particular driver input of the motor vehicle. This ensures that a consent of the driver to assistance in the longitudinal control and/or lateral control of the motor vehicle by means of the driver assistance function is particularly high. As a result, the likelihood that the driver of the motor vehicle utilizes the driver assistance function for controlling the motor vehicle is particularly high.

Ini some examples, an electronic processing device is disclosed, configured to carry out any of the methods for training the software agent by means of the training data. The electronic processing device can thus be configured to receive the generated training data and to train the software agent. The electronic processing device can, for example, be a hardware component on which the software agent can be executed.

In some examples, a motor vehicle is disclosed, comprising a driver assistance system, which is configured to assist a driver in the control of the motor vehicle by means of a driver assistance function. This driver assistance function is adapted using any of the methods described herein for assisting a driver in the control of a motor vehicle by means of a driver assistance function. This driver assistance system can be configured to be adapted by the trained software agent. As an alternative, the software agent can be the driver assistance system or a part thereof and can be configured to directly control the driver assistance function.

### BRIEF DESCRIPTION OF THE DRAWINGS

Further features of the invention can be derived from the following description of the figures and based on drawings. The features and feature combinations mentioned above in the description, and the features and feature combinations shown hereafter in the description of the figures and/or shown only in the figures, can be used not only in the respectively indicated combination, but also in other combinations, or alone, without departing from the scope of the invention.

In the drawings:

FIG. 1 shows a first schematic diagram for a method for generating training data for a software agent and for a method for assisting a driver in the control of a motor vehicle using a driver assistance function, according to some aspects of the present disclosure;

FIG. 2 shows a second schematic diagram for a method for generating training data for a software agent and for a method for assisting a driver in the control of a motor vehicle using a driver assistance function, according to some aspects of the present disclosure;

FIG. 3 shows a coordinate system in which an actually traveled trajectory of a motor vehicle is shown, in a state in which the driver of the motor vehicle was assisted in the control of the motor vehicle using a driver assistance function, wherein additionally a trajectory planned for the motor vehicle by the driver assistance function is plotted, according to some aspects of the present disclosure; and
motor vehicle by the driver assistance function differs from the trajectory from FIG. 3 actually traveled by the motor vehicle, according to some aspects of the present disclosure.

### DETAILED DESCRIPTION

Like or functionally equivalent elements are denoted by like reference numerals in the figures.

As used herein, a software agent—also referred to as an agent or softbot—denotes a computer program that is capable of exhibiting specified, independent, and self-generated behavior. This refers to the performance of certain processing operations based on different internal states, without requiring an external start signal or any control intervention from outside during the operation. A software program may be considered an agent when it exhibits characteristics such as autonomy, cognition, communication, modal adaptability, activity, reactivity, robustness, and/or social interaction. These properties define the degree of autonomy of the computer program. In particular, autonomous behavior is understood to mean that the software agent operates independently of user intervention. Cognitive behavior refers to the software agent being teachable and capable of learning from previously made decisions or observations. Communicative behavior refers to the software agent conveying its internal states to its surroundings, thereby exerting influence. Modal adaptability refers to the software agent modifying its own settings—particularly parameters and/or structure—based on its internal state and the state of its surroundings. Active behavior is understood to mean that the software agent executes actions on its own initiative. Reactive behavior refers to the software agent responding to changes in the surrounding environment. Robustness refers to the software agent compensating for external or internal disturbances. Social behavior refers to the software agent communicating with other agents.

In some examples, a trajectory for the motor vehicle is planned for a detected situation using the driver assistance function. Additionally, a longitudinal and/or lateral control strategy for guiding the motor vehicle along the planned trajectory is determined, and an actuator system of the motor vehicle is set accordingly to implement this control. In other words, the driver assistance function performs a path planning operation for the motor vehicle, and the actuator system—which influences the steering and/or acceleration and/or deceleration of the motor vehicle—is correspondingly adjusted to steer or assist in steering the vehicle along the planned trajectory.

In some examples, a reaction of the driver to the actuator system that has been set is ascertained. In this manner, it is determined how the driver responds to the steering and/or acceleration and/or deceleration influenced by the actuator system. Based on the ascertained reaction of the driver, a divergence—characterized by a divergence value—is determined between the longitudinal and/or lateral control resulting from the driver's reaction (and therefore planned by the driver) and the longitudinal and/or lateral control resulting from the actuator system that was set according to the driver assistance function. Thus, based on the driver's reaction to the actuator system set by the driver assistance function, it is determined whether the driver agrees or disagrees with the longitudinal and/or lateral control as defined by the driver assistance function. The lower the divergence value, the smaller the deviation between the driver's intended control behavior and the control behavior implemented by the actuator system. A low divergence value therefore allows the inference that the driver agrees with the control behavior planned by the driver assistance function. In contrast, a high divergence value suggests that the driver disagrees with the control behavior implemented by the driver assistance function, meaning that the driver's intended control significantly deviates from the automated control applied via the actuator system. The divergence value thus represents the extent to which the driver and the driver assistance function differ in their respective control of the motor vehicle.

In some examples, the driver's reaction to control of the motor vehicle, assisted by the driver assistance function, is directly ascertained. This means that the actuator system is, in all cases, set or adjusted by the driver assistance function. As a result, an analysis of so-called ground truth data—wherein the motor vehicle is operated purely under manual control by the driver—is not necessarily required.

In some examples, the ascertained driving situation is provided, together with the corresponding assigned divergence value, as training data for the software agent. In this manner, the driver of the motor vehicle is supported in controlling the vehicle in the identified situation by way of the driver assistance function. Specifically, a trajectory for the motor vehicle is planned, and the actuator system is adjusted to perform the lateral and/or longitudinal control of the vehicle in accordance with the planned trajectory. The respective divergence value determined for each situation is assigned to the training data as a label, enabling the software agent to be trained particularly effectively using the respective situations—both identical and similar. The generated training data thus allow the software agent to be trained in a manner that reduces the divergence value, meaning that the control assistance provided by the driver assistance function becomes more closely aligned with the expectations or preferences of the driver. The objective of such training is to promote agreement by the driver with the control actions of the driver assistance function. In some examples, the software agent comprises an artificial neural network that is trained using the generated training data.

In some examples, the actuator system is set based on a defined intervention dominance of the driver assistance function. The intervention dominance characterizes the extent to which the driver assistance function asserts the planned longitudinal and/or lateral control for a given trajectory in comparison to the driver's own objectives or actions. In this context, the intervention dominance describes the strength of intervention by the software agent. A higher intervention dominance corresponds to stronger enforcement by the driver assistance function of the planned control actions, while a lower intervention dominance results in weaker assertion relative to the driver's inputs. For instance, at an intervention dominance of 0%, the motor vehicle is controlled entirely manually by the driver. At an intervention dominance of 100%, the vehicle is controlled exclusively by the driver assistance function, with no influence from the driver. In this case, the driver's control is overridden by the system. At intermediate levels of intervention dominance, the driver receives varying degrees of assistance from the driver assistance function. In the present example, the intervention dominance is greater than 0%. The actuator system is configured accordingly, such that the driver assistance function asserts the planned longitudinal and/or lateral control along the trajectory to a greater or lesser extent, depending on the specified intervention dominance and the degree to which the driver's actions are considered. For example, in the case of low intervention dominance for steering, the actuator system may assist the driver by reducing the torque required to steer in the intended direction while increasing it in the opposite direction. In contrast, at high intervention dominance, the driver assistance function may directly apply a steering torque to influence the vehicle's movement. By setting the actuator system in accordance with the defined intervention dominance, the desired level of driver support can be maintained.

In some examples, the driver's reaction is determined based on an actuation of the gas pedal, the brake pedal, and/or a steering device. In this way, the reaction is inferred from the driver's influence over the vehicle's driving direction and/or acceleration, i.e., over the longitudinal and/or lateral guidance of the vehicle. Based on the degree of such input, it is established how the driver responds to the actuator system adjustments carried out by the driver assistance function. The reaction can thus be ascertained in a particularly simple and reliable manner based on the driver's actuation of the gas pedal, brake pedal, and/or steering device.

In some examples, the reaction of the driver is determined based on a measured error-related brain potential in the driver's brain. When a person recognizes an error while performing a task, a characteristic brain potential—referred to as an error-related potential—can be detected as a physiological response. In the present context, this brain potential may be measured, for example, using an electrode cap worn by the driver. Accordingly, in order to determine the driver's reaction, the brain potential is measured while the vehicle is being controlled in an assistance-based manner using the driver assistance function during a particular driving situation. In other words, while the actuator system is configured to assist the driver in controlling the vehicle in a given situation, a corresponding brain potential is detected to determine the driver's response to the assistance. Based on the measured brain potential, it is possible to reliably establish whether the driver agrees with or disagrees with the support provided in the lateral and/or longitudinal guidance of the motor vehicle-without requiring the driver to manually intervene. This physiological signal enables the detection of unconscious agreement or unconscious rejection by the driver regarding the control behavior of the driver assistance function.

In some examples, a divergence value is compared to a specified threshold value. If it is determined that the divergence value is below the threshold, then the corresponding situation is assigned, within the training data, as a case in which no divergence exists. Conversely, if the divergence value is equal to or exceeds the threshold, then the situation is assigned as one in which a divergence is present. Accordingly, the training data may assign a label to each respective situation indicating whether a divergence occurred, for example as “Divergence: yes” or “Divergence: no.” This type of labeling simplifies the training of the software agent using such data.

In some examples, the software agent is trained using reinforcement learning, wherein either the divergence value itself or the frequency of detected divergence is used as a reward metric that is to be minimized. That is, the objective during reinforcement learning is to reduce the divergence value derived from the driver's reaction to the actuator system set by the driver assistance function. Alternatively, or in addition, the objective may be to minimize the number of situations in which a divergence is detected relative to the number of situations in which no divergence is detected, across a variety of operating scenarios in which the actuator system is engaged by the driver assistance function under the control of the software agent. In other words, in scenarios where the driver is repeatedly supported by the software-agent-controlled driver assistance function, the learning objective is to reduce the proportion of situations involving divergence.

Reinforcement learning refers to a category of machine learning methods in which the software agent learns, through interaction with its environment, a strategy that maximizes received rewards. In the present context, the reward is considered to be maximized when the divergence value is particularly low or when the proportion of divergence-labeled situations is reduced. During reinforcement learning, the software agent is not explicitly shown which action or situation is optimal; instead, it receives positive or negative feedback (i.e., rewards) based on the consequences of its behavior at various points in time. In the present example, the software agent may be continuously trained using newly generated training data during active operation of the vehicle. As such, the software agent is iteratively improved over time. The use of reinforcement learning thus allows the driver assistance function, when controlled by the software agent, to be adapted particularly quickly and effectively to the individual preferences of the driver.

In some examples, the software agent is trained using reinforcement learning, wherein either the divergence value or the frequency of divergence serves as a reward metric to be minimized. The training objective is to reduce the divergence value determined based on the driver's reaction to the actuator system that is configured by the driver assistance function. Alternatively, or additionally, the training objective may involve minimizing the frequency of situations in which a divergence is detected, relative to the frequency of situations in which no divergence is detected. This applies across multiple processes carried out in different situations where the actuator system of the motor vehicle is activated via the driver assistance function under the control of the software agent. In this manner, when the driver is supported by the driver assistance function in a variety of situations, the goal of reinforcement learning is to reduce the proportion of situations in which a divergence has been ascertained.

Reinforcement learning refers to a class of machine learning methods in which the software agent learns a strategy to maximize received rewards. In the present context, the reward is maximized when a particularly low divergence value is achieved or when the number of divergence-labeled situations is reduced. During reinforcement learning, the software agent is not explicitly informed which actions or situations are optimal. Rather, it interacts with its environment and receives feedback in the form of rewards or penalties at discrete points in time. In some examples, the software agent may be trained continuously while the motor vehicle is in operation, using newly generated training data to further improve its behavior. The use of reinforcement learning enables the driver assistance function—when controlled by the software agent—to be adapted particularly effectively and efficiently to the driver's preferences.

The present disclosure also relates to a method for assisting a driver in controlling a motor vehicle by means of a driver assistance function. In this method, the driver assistance function is adapted using a software agent that has been trained in accordance with any of the methods described herein for training a software agent. The driver is supported by the adapted driver assistance function, which sets the actuator system of the motor vehicle for a given situation. This represents an end-to-end machine learning approach in which situation-representing camera data is provided to the software agent, and the software agent directly outputs actuator system commands for the motor vehicle. This approach enables control of the motor vehicle via a driver assistance function that is particularly well adapted to the driver's preferences.

In some examples, the driver assistance function is further adapted by the software agent with respect to path planning and/or intervention dominance. The driver is supported in controlling the vehicle by means of the adapted driver assistance function, which, in a given situation, performs trajectory planning, determines a corresponding longitudinal and/or lateral control strategy, and sets the actuator system of the vehicle based on an intervention dominance adapted by the software agent. The software agent may execute the driver assistance function itself, or it may influence a separate driver assistance system. In the latter case, the driver assistance system is distinct from the software agent but may be adjusted by it for purposes of modifying the path planning and/or intervention dominance. The adaptation of path planning and/or intervention dominance by the trained software agent enables the driver assistance function, and thus the assistance provided to the driver, to be closely aligned with the driver's individual inputs.

In some examples, the criticality of the ascertained situation is determined, and the intervention dominance is selected based on the established level of criticality. That is, in more critical situations, a higher intervention dominance is selected so that the driver assistance function intervenes more strongly in the longitudinal and/or lateral control of the motor vehicle. In such cases, the actuator system is set to reduce or prevent risks to the vehicle and its occupants. In less critical situations, the intervention dominance may be selected to be particularly low, allowing the driver to retain greater control over the longitudinal and/or lateral guidance of the motor vehicle. This provides the driver with a heightened sense of control over the vehicle's steering and maneuvering behavior.

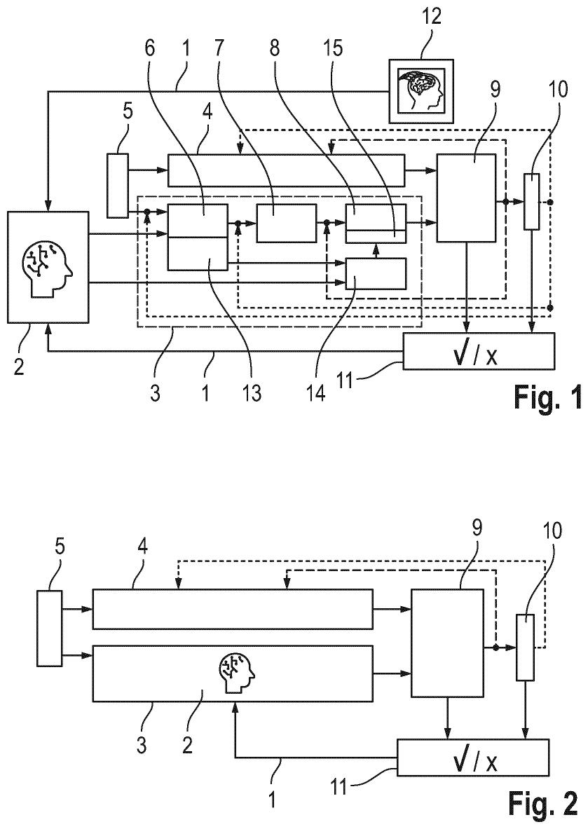

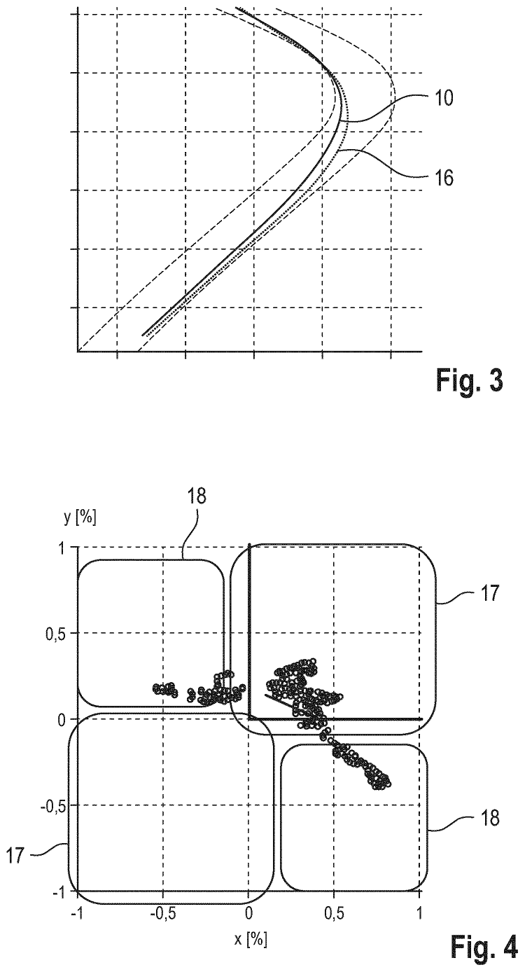

FIG. 1 shows a schematic diagram for a method for generating training data 1 for a software agent 2, which is configured to control a driver assistance function 3 of a motor vehicle. Furthermore, a method for assisting a driver 4 in the control of a motor vehicle by means of a driver assistance function 3 is shown in the schematic diagram. The training data 1 can be generated while the driver 4 is being assisted in the control of the motor vehicle. FIG. 2 likewise shows a schematic diagram for a method for generating training data 1 for the software agent 2, wherein the software agent 2 executes and thus controls the driver assistance function 3. The schematic diagram of the method in FIG. 2 furthermore encompasses the method for assisting the driver 4 in the control of the motor vehicle by means of a driver assistance function 3. The software agent 2 can be configured, as shown in FIG. 1, to adapt the driver assistance function 3. Alternatively, the software agent 2, as shown in FIG. 2, can execute the driver assistance function 3 itself. As is apparent from the schematic diagrams of the methods in FIG. 1 and FIG. 2, the driver 4 can be assisted in the control of the motor vehicle by means of the driver assistance function 3 and, at the same time, the training data 1 for the software agent 2 can be generated.

To assist the driver 4 in the control of the motor vehicle, a situation 5 in which the motor vehicle finds itself is ascertained. This ascertained situation 5 is provided to the driver assistance function 3. Within the scope of path planning 6, a target trajectory 16 is planned for the motor vehicle for the ascertained situation 5 by means of the driver assistance function 3. Thereafter, a driving dynamics control 7 is carried out by means of the driver assistance function 3, within the scope of which a longitudinal control and/or lateral control intended for guiding the motor vehicle along the target trajectory 16 is planned. For example, steering angles for the planned target trajectory 16 can be calculated in the process. The driver assistance function 3 is furthermore configured to subsequently carry out an actuator system control 8. Within the scope of the actuator system control 8, the calculated steering angle is implemented by means of the actuator system of the motor vehicle. Within the scope of the actuator system control 8, the actuator system of the motor vehicle is set for the implementation of the planned longitudinal control and/or lateral control. As a result of the adaptation of the actuator system, the lateral and/or longitudinal control of the motor vehicle can be influenced, in that respective control devices 9 of the motor vehicle are affected. At the same time, a notion may exist in the mind of the driver 4 as to what the trajectory should look like along which the motor vehicle is to be guided in the ascertained situation 5, and what a longitudinal guidance of the motor vehicle along this trajectory should entail. As a result, the driver 4 can influence the lateral and/or longitudinal guidance of the motor vehicle. This influencing of the lateral and/or longitudinal guidance of the motor vehicle by the driver 4, in turn, results in an actual trajectory 10 that is covered by the motor vehicle under a defined speed profile. Based on the setting of control devices 9, which affects the motor vehicle, such as the gas pedal and/or the brake pedal and/or the steering system, as well as based on the actual trajectory 10 traveled by the motor vehicle in the ascertained situation 5, it is possible to ascertain whether a divergence 11 exists between the longitudinal control and/or lateral control of the motor vehicle along the target trajectory 16, as planned by the driver assistance function 3, and a driver input. This divergence 11 is determined based on the reaction of the driver 4 to the actuator system control 8 performed by the driver assistance function 3.

Within the scope of the actuator system control 8, it is possible, for example, for the driver assistance function 3, by adapting respective actuators, to make turning the steering wheel in a first direction easier for the driver 4, while turning it in a second direction opposite the first direction is made more difficult. In this way, the driver 4 can be prompted by means of the driver assistance function 3 to steer in the first direction so that the motor vehicle is guided as much as possible in accordance with the target trajectory 16 planned by the driver assistance function 3. To assist the driver 4 in longitudinal control, at least one actuator, for example, can be set by means of the driver assistance function 3 in such a way that it is particularly easy for the driver 4 to press down on the gas pedal and that there is resistance when pressing the brake pedal, prompting the driver 4 to drive more quickly along the trajectory by accelerating the motor vehicle. Through the actuator system control 8, the driver assistance function 3 can thus prompt the driver 4 to control the motor vehicle in accordance with the longitudinal guidance and/or lateral guidance planned by the driver assistance function 3. By appropriately actuating the steering wheel, gas pedal, or brake pedal, the driver 4 can control the motor vehicle in accordance with or counteract the longitudinal guidance and/or lateral guidance planned by the driver assistance function 3.

To ascertain the divergence 11, the reaction of the driver 4 to the actuator system set by the driver assistance function 3 is evaluated. In doing so, the reaction of the driver 4 in the present example is assessed based on the actuation of the control devices 9 by the driver 4 and/or based on the actual trajectory 10 covered by the motor vehicle in the ascertained situation 5. The divergence 11 can be determined, for example, based on a deviation of the actual trajectory 10 that was covered by the motor vehicle from the target trajectory 16 planned by the driver assistance function 3. The divergence 11 can, in particular, be expressed as a divergence value that characterizes the divergence 11. The divergence 11 describes the deviation between the planned longitudinal control and/or lateral control by the driver assistance function 3 compared to the planned longitudinal control and/or lateral control of the driver 4.

In the method, it is thus established based on the ascertained reaction of the driver 4 how large the divergence 11, characterized by a divergence value, is between the longitudinal control and/or lateral control planned by the driver 4 and the longitudinal control and lateral control of the motor vehicle characterized by the influenced actuator system and planned by the driver assistance function 3, along the planned target trajectory 16. The situation 5 is provided together with the assigned divergence value as training data 1 for the software agent 2. The software agent 2 can, in turn, be trained using the training data 1, in particular through reinforcement learning. In this training method, the specified goal is that a respective divergence value ascertained for further situations 5 is to be particularly low or that it is to be ascertained as rarely as possible for further analyzed situations 5 that a divergence 11 exists.

In the present example, it is provided that the reaction of the driver 4 is additionally ascertained based on a measured error-related brain potential 12. This brain potential 12 can be determined by means of electrodes attached to the head of the driver 4. In particular, it is assessed how the brain potential 12 of the driver 4 reacts to the actual longitudinal guidance and/or lateral guidance of the motor vehicle in the ascertained situation 5. Based on the measured error-related brain potential 12, it is possible, in turn, to determine the divergence 11 characterized by the divergence value. The situation 5 can be provided together with the divergence value, which was ascertained based on the brain potential 12, as training data 1 for the software agent 2. The error-related brain potentials, which can also be referred to as error potentials, are generated automatically by the brain when the real world deviates from one's own expectations. These can be measured by a brain-computer interface. If the driver assistance function 3 does not perform as the driver 4 expects, the passive but measurable error potential signal can be recorded.

Within the scope of the path planning 6, a safety prediction 13 can be made. In this safety prediction 13, the criticality of the situation 5 in combination with the target trajectory 16, ascertained within the scope of the path planning 6, is determined. This means that it is assessed how likely a risk of damage, particularly a collision of the motor vehicle with another object, is in the situation 5, and whether this risk can be averted if the motor vehicle follows the target trajectory 16 created within the scope of the path planning 6. An intervention dominance 14 of the driver assistance function 3 is adapted based on this ascertained criticality of the situation 5. This intervention dominance 14 determines how strongly the control of the motor vehicle is to be influenced by the driver assistance function 3 and how strongly it is to be influenced by the driver 4. Based on the set intervention dominance 14, the actuator system controlled by the actuator system control 8 can be adapted by means of an adaptation control 15, whereby the longitudinal guidance and/or lateral guidance of the motor vehicle is influenced to a greater or lesser degree by the driver assistance function 3 in accordance with the level of the intervention dominance 14.

It is provided in the method shown in FIG. 1 that the path planning 6, the safety prediction 13, and/or the intervention dominance 14 are adapted by means of the trained software agent 2. By adapting the safety prediction 13, the software agent 2 can specify what level of criticality applies to which types of situations 5. By influencing the intervention dominance 14, it can be determined by the software agent 2 at what ascertained criticality of respective situations 5 the driver assistance function 3 is to be granted what level of dominance over the driver 4.

The path planning 6 and/or the intervention dominance 14 of the driver assistance function 3 are thus adapted by means of the software agent 2, which has been trained based on the training data 1. The driver 4 is subsequently assisted in the control of the motor vehicle by means of the adapted driver assistance function 3. During this assistance, a target trajectory 16 is planned for the motor vehicle within the scope of the adapted path planning 6 by means of the driver assistance function 3 for the ascertained situation 5. A longitudinal control and/or lateral control intended for guiding the motor vehicle along the target trajectory 16 is planned, and the actuator system of the motor vehicle is set by means of the actuator system control 8 based on the adapted intervention dominance 14 for implementing the planned longitudinal control and/or lateral control.

To assign particularly simple labels to the respective situations 5 in the training data 1, it may be provided that the ascertained divergence value characterizing the divergence 11 is compared to a specified threshold value, and in the training data 1, it is assigned to the situation 5 that no divergence 11 exists if it is established that the divergence value is smaller than the specified threshold value. If it is established that the divergence value is greater than or equal to the specified threshold value, it is assigned in the training data 1 to the situation 5 that a divergence 11 exists. The training data 1 are thus merely labeled “Divergence: yes” or “Divergence: no” for the particular situation 5.

In the embodiment of the method shown in FIG. 2, the software agent 2 is configured to execute the driver assistance function 3. The software agent 2 is trained analogously to the method that was already described in connection with FIG. 1 based on the training data 1, in which the associated ascertained divergence 11 is assigned to respective ascertained situations 5 in the form of the divergence value or in the form of “Divergence exists”/“Divergence does not exist” as a label. It is provided in the method shown in FIG. 2 that the path planning 6, the driving dynamics control 7, and/or the actuator system control 8 are carried out by means of the software agent 2. In addition, the safety prediction 13 and the adaptation of the intervention dominance 14 can be carried out by means of the software agent 2, and the adaptation of the actuator system specified by the actuator system control 8 can be carried out based on the intervention dominance 14 by means of the adaptation control 15.

The method for assisting driver 4 in the control of the motor vehicle can, for example, be used within the scope of a lane-keeping assistance system serving as the driver assistance function 3. A steering torque can thus be applied by means of the driver assistance function 3, wherein the steering of the motor vehicle is performed by driver 4.

FIG. 3 shows the actual trajectory 10 traveled by the motor vehicle compared to the target trajectory 16 planned by the driver assistance function 3 over the progression of a curve. It can be recognized here that the actual trajectory 10 traveled by the motor vehicle deviates from the planned target trajectory 16. A divergence 11 thus exists between the planning of the lateral control of the motor vehicle by the driver assistance function 3, expressed by the target trajectory 16, and the lateral guidance of the motor vehicle requested by driver 4, which, in combination with the actuator system set by the driver assistance function 3, results in the actual trajectory 10.

In FIG. 4, the assistance moment y applied by the driver assistance function 3 is plotted on the y-axis, and the driver moment x applied by driver 4 is plotted on the x-axis in percent, wherein the respective moments are indicated in percent and normalized to the value of the input and thus normalized to the measurement range of the respective sensor detecting the moment. Two convergence regions 17 as well as two divergence regions 18 are present in the coordinate system. The convergence regions 17 characterize situations in which the same or a similar moment is applied by driver 4 as by the driver assistance function 3. The moments applied by driver 4 deviate from the moments applied by the driver assistance function 3 in the respective divergence regions 18. Depending on whether the measurement values measured at a point in time at the respective sensors yield a point in the diagram that is located in one of the divergence regions 18 or in one of the convergence regions 17, it can be established that a divergence 11 exists when the point is located in one of the divergence regions 18, and that no divergence 11 exists when the point is located in one of the convergence regions 17. Additionally, a deviation of the actual trajectory 10 from the target trajectory 16 planned by the driver assistance function 3 can be included in this correlation analysis between the driver moment x and the assistance moment y. In particular, a deviation direction may be included here.

A divergence 11 between driver 4 and the driver assistance function 3 occurs, in particular, in the path planning 6 and/or in the actuator system control 8. Within the scope of the method, a conflict that is measurable in physical signals is measured and used for adapting the driver assistance function 3. Within the scope of the path planning 6 and the actuator system control 8, many conflicts can arise, for example, due to deviating planning or due to unfavorable intervention strategies. The divergences 11 can arise, for example, when driver 4 does not approve of or did not expect an action of the driver assistance function 3, or when the driver assistance function 3, despite an identical planned trajectory, implements it in a way that seems strange to driver 4, for example, too rigidly.

As an additional statistical analysis option for the assessment of divergence 11, a mean value and a covariance matrix can be used, particularly for supervised learning approaches for subsequent analysis and adaptation, or as an episode reward for a reinforcement learning agent.

Since the introduction of assistance systems, endeavors have been made to reduce the divergence 11 between the driving style of the driver 4 and the situation-based decisions of the system. Often, direct input from the driver 4 regarding his or her requests is used for this purpose, or a larger volume of data is needed to infer user profiles. This approach has the disadvantage that manual parameterization can only be implemented for a very small number of easy-to-understand parameters, such as a time gap in adaptive cruise control systems. Additionally, observing the driver to generate data for the user profile is only meaningful during manual driving, as the driving styles of the driver 4 and the driver assistance function 3 are blended during assisted driving. It is unclear whether the data from manual driving correspond to the requests of the driver 4 when he or she is assisted in the driving operation. Just as one does not want to be chauffeured as a passenger in the same manner as one would personally drive, it is also possible in assisted mode that assistance using one's own driving style may not be desired.

If assistance interventions diverge from the personal or subjective decisions of the using driver 4, this can result in a poor rating all the way to deactivation or non-use of driver assistance functions 3. If the driver 4 behaves differently than the driver assistance function 3, the preferred actions regarding the steering wheel, gas pedal and brake diverge from one another. It is provided in the method for generating the training data 1 that this divergence 11 between the driver 4 and the driver assistance function 3 is quantified as a characteristic value, in the present example as a divergence value, and this divergence 11 is used as input for a machine learning method. Both supervised learning methods and reinforcement learning methods can use this feature of divergence 11 as a label or as a negative reward function so as to successively adapt actions of the driver assistance function 3 in such a way that these result to a lesser degree and less frequently in divergence 11 from the driver input and, by taking the system request of the driver assistance function 3 into consideration, nonetheless a change or an improvement in the driving style of the driver 4 is effectuated. This allows an automated adaptation of the driver assistance function 3, which additionally does not require a manual driving operation for data acquisition, and furthermore is able to continue to learn if the driver 4 is already accepting assistance. The latter is possible since the software agent 2 does not learn from the observed driving style, which in the assisted mode is a mixture of the driver 4 and the driver assistance function 3, but from recognizing divergence 11 or also non-existent divergence 11 between the driver 4 and the driver assistance function 3. It is thus possible to achieve, as a result of the method, that the driver assistance function 3 adapts step by step to ideal assistance notions of the driver 4.

The divergence 11 can be ascertained between specific activities on the steering wheel, gas pedal and brake pedal or as a direct divergence value or as a divergence 11 between the notions of the driver assistance function 3 and the driver 4, as indirect or acausal characteristic values.

The driver assistance function 3 and the driver 4 can mutually perceive one another via actions on the interfaces of the motor vehicle. A divergence 11 between a driver action of the driver 4 and a system action of the driver assistance function 3 primarily arises due to differing notions for the future. As a result of control parameters that are implemented differently in the actuator system control 8, the driver assistance function 3 introduces an additional interaction component, which can be considered to be positive or negative. One example of this is how strong the moment is which the driver assistance function 3 at a maximum may use to assist the driver 4.

The divergence 11 can be calculated based on characteristic values that are based on the actions of the motor vehicle that are ultimately carried out. Additional characteristic values can be calculated by way of correlation and covariance formation of a longer measurement series and can be utilized for adaptation, in addition to assessment of every point in time. Based on the correlation of the driver steering torque and of the assistance steering torque, a distinction can be made as to whether the driver 4 simply just passively holds the steering wheel or works actively against the recommendations of the driver assistance function 3. An actively intervening driver 4 can confirm the actions of the driver assistance function 3 or counteract these.

Different ways for calculating divergence features exist. These can also be referred to as inverse acceptance features or as contradiction determination. These divergence features can adapt any arbitrary design of an automated system, in particular also systems that from start to end are made up of artificial neural networks and no longer have any clearly interpretable structure, as shown in FIG. 2. The error-related brain potentials can be introduced as an additional marker into the system adaptation and thus be used for determining the labels of the respective situations 5 in the training data 1. The software agent 2 can have undergone prior training based on data collected during a manual driving operation of the motor vehicle by the driver 4. Influences of the adaptation, and thus of the adaptation of the driver assistance function 3 by the software agent 2, can be clearly detected in the method and provided with boundaries. In particular, it is provided that the software agent 2 only adapts a parameterization of the algorithms underlying the driver assistance function 3. For example, the software agent 2 could learn that no divergences 11 arise if it never intervenes. It is thus possible to specify for the adaptation of the driver assistance function 3 or for the control of the motor vehicle by means of the driver assistance function 3 that an intervention of the driver assistance function 3 within the scope of the actuator system control 8 must not be smaller than a specified threshold value so as to prevent this case.

The method makes it possible for the driver assistance function 3 to be continuously adapted to the driver 4, without active programming of the driver, and becomes increasingly better during ongoing operation of the motor vehicle.

Overall, the invention shows how a use of divergence features can be implemented for adapting learning algorithms for assisted driving functions.

### List of Reference Numerals

1 training data
2 software agent
3 driver assistance function
4 driver
5 situation
6 path planning
7 driving dynamics control
8 actuator system control
9 control device
10 actual trajectory
11 divergence
12 error-related brain potential
13 safety prediction
14 intervention dominance
15 adaption control
16 target trajectory
17 convergence region
18 divergence region

1-10. (canceled)

11. A method for generating training data for a software agent configured to control a driver assistance function of a motor vehicle, the method comprising:
planning, by the driver assistance function, a trajectory for the motor vehicle based on an ascertained driving situation;
planning a longitudinal control and/or a lateral control for guiding the motor vehicle along the planned trajectory;
setting an actuator system of the motor vehicle to implement the planned longitudinal control and/or lateral control;
ascertaining a reaction of a driver to the actuator system that has been set;
determining a divergence value representing a difference between a longitudinal control and/or lateral control corresponding to the driver's reaction and the longitudinal control and/or lateral control implemented by the actuator system; and
providing the ascertained driving situation together with the divergence value as training data for the software agent.

12. The method of claim 11, wherein the actuator system is set based on a specified intervention dominance of the driver assistance function.

13. The method of claim 11, wherein the reaction of the driver is ascertained based on actuation of a gas pedal, a brake pedal, or a steering device.

14. The method of claim 11, wherein the reaction of the driver is ascertained based on a measured error-related brain potential of the driver.

15. The method of claim 11, further comprising comparing the divergence value to a threshold value and assigning, based on the comparison, a label to the ascertained driving situation indicating whether a divergence is present.

16. The method of claim 11, further comprising training the software agent using the training data, wherein the training comprises reinforcement learning that adjusts parameters of the software agent to reduce either a magnitude of the divergence value or a frequency of divergence events across a plurality of driving situations.

17. The method of claim 16, wherein the training data further comprises labels generated based on error-related brain potentials of the driver indicating implicit disagreement with assistance provided by the driver assistance function.

18. A method of assisting a driver in controlling a motor vehicle, comprising:
training a software agent using training data comprising, for each of a plurality of driving situations, a divergence value representing a difference between a control action planned by the driver and a control action implemented by a driver assistance function;
adapting the driver assistance function using the trained software agent; and
assisting the driver in controlling the motor vehicle by using the adapted driver assistance function to set an actuator system of the motor vehicle based on an ascertained driving situation.

19. The method of claim 18, wherein the training data further comprises one or more labels generated based on error-related brain potentials of the driver.

20. The method of claim 18, wherein adapting the driver assistance function comprises modifying a path planning algorithm of the driver assistance function using one or more parameters determined by the software agent.

21. The method of claim 18, wherein adapting the driver assistance function comprises adjusting an intervention dominance level based on one or more outputs of the software agent.

22. The method of claim 18, further comprising planning a trajectory for the motor vehicle using the adapted driver assistance function, and planning a longitudinal control and/or a lateral control for guiding the motor vehicle along the planned trajectory.

23. The method of claim 22, further comprising setting the actuator system of the motor vehicle to implement the planned longitudinal control and/or lateral control based on the adjusted intervention dominance level.

24. The method of claim 18, wherein the software agent is trained using reinforcement learning that minimizes the divergence value or a frequency of divergence events.

25. A motor vehicle comprising:
a driver assistance system comprising a software agent trained using training data comprising, for each of a plurality of driving situations, a divergence value representing a difference between a control action planned by a driver and a control action implemented by a driver assistance function;
wherein the driver assistance system is configured to adapt the driver assistance function using the trained software agent; and
wherein the adapted driver assistance function is configured to set an actuator system of the motor vehicle for an ascertained driving situation to assist the driver in controlling the motor vehicle.

26. The motor vehicle of claim 25, wherein the training data further comprises one or more labels generated based on error-related brain potentials of the driver.

27. The motor vehicle of claim 25, wherein the driver assistance system is configured to adapt a path planning module of the driver assistance function based on the training data.

28. The motor vehicle of claim 25, wherein the driver assistance system is configured to adjust an intervention dominance level of the driver assistance function based on the training data.

29. The motor vehicle of claim 25, wherein the adapted driver assistance function is further configured to plan a trajectory and a longitudinal control and/or a lateral control for guiding the motor vehicle along the trajectory.

30. The motor vehicle of claim 29, wherein the actuator system is configured to implement the planned longitudinal control and/or lateral control based on the adjusted intervention dominance level.
